SEQUENCE LISTING (4 sequences)

SEQ ID NO: 1 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 2 - protein, 1295 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIGELVFQLAWKPVV

SEQ ID NO: 3 - DNA, 5984 bp, Homo sapiens
acaaaaggtcctgtgctcacctgggacccttctggacgttgccctgtgttcctcttcgcctgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcatccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcatgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattgccaaagtagcaatcagtggaggtttccagaaacttgagaacctaaagctttcaatcaatcacaagattacagaggaaggatacagaaatttctttcaagcactggacaacatgccaaacttgcaggagttggacatctccaggcatttcacagagtgtatcaaagctcaggccacaacagtcaagtctttgagtcaatgtgtgttacgactaccaaggctcattagactgaacatgttaagttggctcttggatgcagatgatattgcattgcttaatgtcatgaaagaaagacatcctcaatctaagtacttaactattctccagaaatggatactgccgttctctccaatcattcagaaataaaagattcagctaaaaactgctgaatcaataatttgtcttggggcatattgaggatgtaaaaaaagttgttgattaatgctaaaaaccaaattatccaaaattattttattaaatattgcatacaaaagaaaatgtgtaaggcttgctaaaaaacaaaacaaaacaaaacacagtcctgcatactcaccaccaagctcaagaaataaatcatcaccaatacctttgaggtccctgagtaatccaccccagctaaaggcaaacccttcaatcaagtttatacagcaaaccctccattgtccatggtcaacagggaaggggttggggacaggtctgccaatctatctaaaagccacaatatggaagaagtattcaatttatataataaatggctaacttaacggttgaatcactttcatacatggatgaaacgggtttaacacaggatccacatgaatcttctgtgggccaaaatatgttccttaatccttgtagaacctgtcttctatattgaactagctttggtacagtagagttaacttactttccatttatccactgccaatataaagaggaaacaggggttagggaaaaatgacttcattccagaggcttctcagagttcaacatatgctataatttagaattttcttatgaatccactctacttgggtagaaaatattttatctctagtgattgcatattatttccatatcatagtatttcatagtattatatttgatatgagtgtctatatcaatgtcagtgtccagaatttcgttcctaccagttgagtagttttctgaacggccagaagaccattcgaaattcatgatactactataagttggtaaacaaccatacttttatcctcatttttattctcactaagaaaaaagtcaactcccctccccttgcccaagtatgaaatatagggacagtatgtatggtgtggtctcatttgtttagaaaaccacttatgactgggtgcggtggctcacacctgtaatcccagcactttgggaggctgaggcgggcgaatcatttgaggtgaggagttcgagaccggcctggccagcatggtgaaaccccatttttgctaaaggtacaaaaattagccaggtgtggtggcacatgcctgtggtcccagccactggggcggctgagacgcaggacttgcttgaacccgggaggcagaggttgcagtgagccgagatggcgccactgcattccagcctgggcaacagagcaagaccctgtctgtttcaaaacaaaaaacaaaaccacttatattgctagctacattaagaatttctgaatatgttactgagcttgcttgtggtaaccatttataatatcagaaagtatatgtacaccaaaacatgttgaacatccatgttgtacaactgaaatataaataattttgtcaattatacctaaataaaactggaaaaaaaaaaaaaaaaaaaaaaaaaaaaaaaaa

SEQ ID NO: 4 - DNA, 5366 bp, Homo sapiens
acaaaaggtcctgtgctcacctgggacccttctggacgttgccctgtgttcctcttcgcctgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcatccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcatgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattggtgagctagtgtttcagcttgcatggaagccagtggtatagccaagctttctgctgcaacatgtctatgtaaacatttgcccctctagaaattttcaacccgcttcctcattttcactatcatactgttccttctagtgtccttctgtggatttaggcgcattctggtcagatttggaagtacaaaaaggtctcccatttgtggatatacaagccctcaaatctgcgttcttgccacctggtgttttagacacctggccacatactctcctaagtactcctttttaaaactgaagatgaatatacacacagaaaagtacaaaaatcatgtgtactgctcactgaattttattttcttattttcttcttttttttttttttgagacagagtttcgctcgtgttgcccaggctggagtacaatggcacgatctcgggtcactgcaaactctgcctcctgggttcaagcgattctcctgcctcagcctcccaagtagctaggattacaggtgaacgccaccacacctggctaattttgtatttttagtaaacacagggtttcaccatgttggccaggctagtctcgaactcctgacctcaagtgagccacagtgcctggcctgaggaactgagatttctgtcgagacctgaagggagaatggcccaggcatagttggtagaggaggaattgagacatcatttcaaacagaggtaatcacttgtgtcatagcctggagttaaagagaaccagatatatttgaagaacttgggggaaaaaaaggaatgtctggagcaagaggcaggagtgagttgtgagaagaagactggagaggaaagtaaaagcccaattggagaggctttgtcgggtgtgttacaagggctggatctcattttcttactgctcagcactgttattttacgttatttaaaacagctgggagcggtggctcaagcttgtaatcccagcactttgggaggccgaggcggatggatcacgaggtcaggagatcgagaccatcctggctaacatggtgaaaccccgtctctactaaaaatacaaaaaattagccaggcgtgatggcgggcacctgtagtcccagctactcgggaggctgaggcaggagaatggtgtgaacccgggaggtggagcttgaagtgagccaagatcatgccactgcactccagcctgggcaacagaacgagactccgtctcaaaaaaaaaaaacaaaaa

(19) United States
(12) Patent Application Publication
(10) Pub. No.: US 2005/0261306 A1
(43) Pub. Date: Nov. 24, 2005

(54) Anti-neurodegenerative agents

(76) Inventors: Joh-E Ikeda, Tokyo (JP); Yoshinori Okada, Kanagawa (JP); Harumi Sakai, Kanagawa (JP); Hitoshi Osuga, Kanagawa (JP)

Correspondence Address: FOLEY AND LARDNER;SUITE 500, 3000 K STREET NW, WASHINGTON, DC, 20007, US

(21) Appl. No.: 11/091,928

(22) Filed: Mar. 29, 2005

Related U.S. Application Data
(63) Continuation-in-part of application No. PCT/JP03/12540, filed on Sep. 30, 2003.

(30) Foreign Application Priority Data
Sep. 30, 2002 (JP) JP 2002-286400

(51) Int. Cl. A61K 31/496; A61K 31/495; A61K 31/4747; A61K 31/454; A61K 31/135; A61K 31/16

(52) U.S. Cl. 514/253.04; 514/255.03; 514/326; 514/278; 514/625

(57) ABSTRACT

The present invention provides methods for treating or preventing neurodegenerative diseases such as amyotrophic lateral sclerosis (ALS), spinal muscular atrophy (SMA), Huntington's disease, Parkinson's disease, Alzheimer's disease, dementia after cerebral vascular disorder, dementia accompanied by other neuronal degeneration. The present invention provides methods for treating or preventing neurodegenerative diseases comprising administering a compound that upregulates neuronal apoptosis inhibitory protein (NAIP) production. Furthermore, the present invention provides methods for treating or preventing neurodegenerative diseases comprising administering one or more compounds selected from the group consisting of: 3-[4-(4-chlorophenyl) piperazin-1-yl] methyl]-1H-pyrrolo [2,3-b] pyridine or salts thereof 5-(4-chlorophenyl)-4-methyl-3-(1-(2-phenylethyl) piperidin-4-yl) isoxazole or salts thereof, 3-(4-chlorophenyl)-4-methyl-5-(1-(2-phenylethyl) piperidin-4-yl) isoxazole or salts thereof, N-methyl-4-(2-cyanophenyl) piperazinyl-3-methylbenzamine or salts thereof, 8-[(2,3-Dihydo-1,4-benzodioxin-2-yl)methyl]-1-phenyl-1,3,8-triazaspiro[4,5]decan-4-one or salts thereof, (E)-N-[(4-Hydroxy-3-methoxyphenyl)methyl]-8-methyl-6-nonenamide or salts thereof, (Z)-N-[(4-Hydroxy-3-methoxyphenyl)methyl]-8-methyl-6-nonenamide or salts thereof, an 5-[[4-[(1-Methylcyclohexyl)methoxy]phenyl]methyl]-2,4-thiazolidinedione or salts thereof. Moreover, the present invention provides methods of screening for an anti-neurodegenerative agent, comprising the steps of: (a) contacting a test sample with a cell and measuring NAIP production; and, (b) selecting a compound that increases the NAIP production in comparison with a control test in which the test sample is not contacted with the cell. Furthermore, the present invention provides compounds that upregulate NAIP production, wherein the compound can be isolated by the above screening method.

This application is a continuation-in-part of PCT/JP2003/012540 (WO 2004/028540), filed Sep. 30, 2003, which claims priority fin Japanese Application 2002-286400, filed Sep. 30, 2002. All of these applications are incorporated herein by reference.

### TECHNICAL FIELD

The present invention relates to methods for treating and preventing neurodegenerative diseases such as amyotrophic lateral sclerosis (ALS), spinal muscular atrophy (SMA), Huntington's disease, Parkinson's disease, Alzheimer's disease, dementia after cerebral vascular disorders, and dementia accompanied by other neuronal degeneration.

### BACKGROUND ART

The group of diseases involving neural cell group degeneration, such as amyotrophic lateral sclerosis (ALS), spinal muscular atrophy (SMA), Huntington's disease, Parkinson's disease, Alzheimer's disease, dementia caused by cerebral vascular disorders, and dementia accompanied by other neuronal degeneration, is generally referred to as neurodegenerative diseases. Fundamental methods of treatment have not been established for most neurodegenerative diseases, and thus treatment methods are being sought.

One approach to treating neurodegenerative diseases is considered to be the administration of factors that suppress neural cell degeneration. Administration of factors that suppress neurodegeneration is expected to be effective in treating and preventing these diseases. However, as yet virtually no such factors have been found to be actually applicable as effective therapeutic drugs.

As the factors that suppress neural cell degeneration, for example, certain dopamine receptor agonists are known to possibly have such a suppression function. However the causal relationship between dopamine antagonists and the suppression of neural cell degeneration is unclear. Moreover, not all dopamine receptor agonists have this effect. In addition, to obtain substances effective as therapeutic drugs, the discovery of other classes of substances that can be used as anti-neurodegenerative drugs is also being sought.

### DISCLOSURE OF THE INVENTION

The present inventors isolated the neuronal apoptosis inhibitory protein (NAIP) gene, a causative gene of the familial hereditary disease SMA, from the human chromosome 5q13.1 region (Roy N et al., Cell 80:167-178,1995). The present inventors also identified the entire amino acid sequence of NAIP, and isolated cDNAs encoding NAIP (Unexamined Published Japanese Patent Application No. (JP-A) Hei 11-116599). Moreover, the present inventors discovered that compounds that upregulate NAIP production were indeed able to suppress neurodegeneration, thus completing the present invention.

Specifically, the present invention provides methods for treating or preventing neurodegenerative diseases comprising administering a compound that upregulates neuronal apoptosis inhibitory protein (NAIP) production. Furthermore, the present invention provides methods for treating or preventing neurodegenerative diseases that comprise administering one or more compounds selected from the group consisting of:
3-[4-(<4-chlorophenyl)piperazin-1-yl]methyl]-1H-pyrrolo[2,3-b]pyridine (hereinafter referred to as “compound 1”) or its salts, 5-(4-chlorophenyl)-4-methyl-3-(1-(2-phenylethyl) piperidin 4-yl) isoxazole (hereinafter referred to as “compound 2”) or its salts,
3-(4-chlorophenyl)-4-methyl-5-(1-(2-phenylethyl) piperidin-4-yl) isoxazole (hereinafter referred to as “compound 3”) or its salts, N-methyl-4-(2-cyanophenyl) piperazinyl-3-methylbenzamine (hereinafter referred to as “compound 4”) or its salts,
8-[(2,3-Dihydro-1,4-benzodioxin-2-yl)methyl]-1-phenyl-1,3,8-triazaspiro[4,5]decan-4-one (spiroxatrine; hereinafter referred to as “compound 5”) or its salts,
(E)-N-[(4-Hydroxy-3-methoxyphenyl)methyl]-8-methyl-6-nonenamide ((E)-capsaicin; hereinafter referred to as “compound 6”) or its salts,
(Z)-N-[(4-Hydroxy-3-methoxyphenyl)methyl]-8-methyl-6-nonenamide ((Z)-capsaicin; hereinafter referred to as “compound 7”) or its salts, and
5-[[4[(1-Methylcyclohexyl)methoxy]phenyl]methyl]-2,4-thiazolidinedione (ciglitazone; hereinafter referred to as “compound 8”) or its salts.

Moreover, the present invention provides methods of screening for anti-neurodegenerative agents, comprising the steps of: (a) containing a test sample with a cell and measuring NAIP production; and, (b) selecting a compound that increases the NAIP production in comparison with a control test in which the test sample is not contacted with the cell. Furthermore, the present invention provides compounds that upregulate NAIP production, wherein the compound can be isolated by the above screening method.

More specifically, the present invention provides the following:
(1) A method for treating or preventing a neurodegenerative disease comprising administering a compound that upregulates neuronal apoptosis inhibitory protein (NAIP) production.
(2) The method of (1), wherein the compound that upregulates neuronal apoptosis inhibitory protein (NAIP) production is selected from the group consisting of: a dopamine receptor antagonist, a serotonin receptor antagonist, a vanilloid receptor agonist, a peroxisome proliferators-activated receptor (PPAR)-γ agonist, and a combination thereof.
(3) The method of (2), wherein the dopamine receptor antagonist is a dopamine D4 antagonist.
(4) The method of (1), wherein the compound that upregulates neuronal apoptosis inhibitory protein (NAIP) production is selected from the group consisting of: a dopamine D4 antagonist, a dopamine D4 agonist, a serotonin 1A antagonist, and a combination thereof.
(5) The method of (3), wherein the dopamine D4 antagonist is selected from the group consisting of: 3-[4-(4-chlorophenyl) piperazin-1-yl]methyl]-1H-pyrrolo[2,3-b]pyridine or salts thereof, 5-(4-chlorophenyl)-4-methyl-3-(1-(2-phenylethyl) piperidin-4-yl) isoxazole or salts thereof, 3-(4-chlorophenyl)-4-methyl-5-(1-(2-phenylethyl) piperidinyl-4-yl) isoxazole or salts thereof, and a combination thereof.
(6) The method of (4), wherein the dopamine D4 agonist is N-methyl-4-(2-cyanophenyl) piperazinyl-3-methylbenzamine or salts thereof.
(7) The method of (4), wherein the serotonin 1A antagonist is 8-[(2,3-Dihydro-1,4-benzodioxin-2-yl)methyl]-1-phenyl-1,3,8-triazaspiro[4,5]decan-4-one (spiroxatrine) or salts thereof.
(8) The method of (2), wherein the vanilloid receptor agonist is selected from the group consisting of: (E)-N-[(4-Hydroxy-3-methoxyphenyl)methyl]-8-methyl-6-nonenamide (E-capsaicin) or salts thereof, (Z)-N-[(4-Hydroxy-3-methoxyphenyl)methyl]-8-methyl-6-nonenamide (Z-capsaicin) or salts thereof, and a combination thereof.
(9) The method of (2), wherein the peroxisome proliferators-activated receptor (PPAR)-γ agonist is 5-[[4-[(1-Methylcyclohexyl)methoxy]phenyl]methyl]-2,4-thiazolidinedione (ciglitazone) or salts thereof.

(10) A method for treating or preventing a neurodegenerative disease comprising administering one or more compounds selected from the group consisting of: 3-[4-(4-chlorophenyl) piperazin-1-yl]methyl]-1H-pyrrolo[2,3-b]pyridine or salts thereof, 5-(4-chlorophenyl)-4-methyl-3-(1-(2-phenylethyl) piperidin-4-yl) isoxazole or salts thereof, 3-(4-chlorophenyl)-4-methyl-5-(1-(2-phenylethyl) piperidin-4-yl) isoxazole or salts thereof, N-methyl-4-(2-cyanophenyl) piperazinyl-3-methylbenzamine or salts thereof, 8-(2,3-Dihydro-1,4-benzodioxin-2-yl)methyl]-1-phenyl-1,3,8-triazaspiro[4,5]decan-4-one or salts thereof, (E)-N-[(4-Hydroxy-3-methoxyphenyl)methyl]-8-methyl-6-nonenamide or salts thereof, (z)-N-[(4-Hydroxy-3-methoxyphenyl)methyl]-8-methyl-6-nonenamide or salts thereof, and 5 [[4-[(1-Methylcyclohexyl)methoxy]phenyl]methyl]-2,4-thiazolidinedione or salts thereof.
(11) A method of screening for an anti-neurodegenerative agent, comprising the steps of
(a) contacting a test sample with a cell and measure neuronal apoptosis inhibitory protein (NAIP) production; and,
(b) selecting a compound that increases the NAIP production in comparison with a control test in which the test sample is not contacted with the cell.
(12) The method of (11), wherein the neuronal apoptosis inhibitory protein NAIP) production is measured by DNA microarray oligonucleotide microarray, protein array, northern blotting, RNase protection assay, western blotting, or reverse transcription polymerase-chain reaction.
(13) A compound that upregulates neuronal apoptosis inhibitory protein (WASP) production, wherein the compound can be isolated by the method of (11) or (12).

In the present invention, “degenerative disease” means a disorder involving degeneration of the central nerve cells, such as amyotrophic lateral sclerosis (ALS), spinal muscular atrophy (SMA), Huntington's disease, Parkinson's disease, Alzheimer's disease, dementia after cerebral vascular disorder, and dementia accompanied by other neuronal degeneration. “Patients” mean human and non-human mammals (particularly useful mammals such as livestock and pets) that develop a neurodegenerative disease.

In the present invention, “neuronal apoptosis inhibitory protein (NAIP)” preferably refers to a protein comprising the amino acid sequence encoded by the nucleotide sequence of SEQ ID NOs: 3, 4, or that disclosed in Roy N et al. (Cell 80: 167-178, 1995), however, not limited thereto. For example, “NAIP” also refers to a protein comprising the amino acid sequence encoded by the nucleotide sequence of SEQ ID NOs: 3, 4, or that disclosed in Roy N et al., in which one or more amino acid residues have been added, deleted, and/or substituted, but is functionally equivalent to the protein described in SEQ D NO: 1 or 2.

An amino acid residue is preferably mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, HK, K S. T), and amino acids comprising the following side chains: aliphatic side-chains (G A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K. H); and aromatic-containing side-chains (H, F, Y, W). (The letters within parenthesis indicate the one-letter amino acid codes.) Amino acid substitutions within each group are called conservative substitutions. It is well known that a polypeptide comprising a modified amino acid sequence in which one or more amino acid residues is deleted, added, and/or substituted can retain the original biological activity (Mark D F et al., Proc Natl Acad Sci USA 81: 5662-5666, 1984; Zoller M J and Smith M, Nucl Acids Res 10: 6487-6500, 1982; Wang A et al., Science 224: 1431-1433, 1984; Dalbadie-McFarland G et al., Proc Natl Acad Sci USA 79: 6409-6413, 1982). The number of mutated amino acids is not limited, but in general, the number ills within 40% of amino acids of each CDR, and preferably within 35%, and more preferably within 30% (e.g., within 25%).

The identity of one nucleotide sequence or amino acid sequence to another can be determined using the algorithm BLAST, by Karlin S and Altschul S F (Proc Natl Acad Sci USA, 90:5873-5877, 1993). Programs such as BLASTN and BLASTX were developed based on this algorithm (Altschul et al., J Mol Biol 215:403-410, 1990). To analyze nucleotide sequences according to BLASTN based on BLAST, the parameters are set, for example, as score=100 and wordlength=12. Similarly, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST)).

Herein, the term “fictionally equivalent” means that the target protein has an activity of suppressing cell-death or the gene of which is causative of the familial hereditary disease SMA. One of ordinary skill in the art can readily know whether a protein suppresses cells or not by, for example, incubating cells with a cell-death inducing agent after treating the cells with or without the protein and comparing the viable cell count after the incubation. The protein is decided to suppress cell-death when the viable cell count after the treatment with the protein is greater than that after the treatment without the protein. In case of the condition of Example 3, the difference of relative fluorescence between two conditions, with the treatment in the presence or absence of the protein, is preferably 3 or more, more preferably 5 or more, and much more preferably 10 or more.

The upregulation or the increase of NAP production can be detected by directly measuring the amount of NAIP itself or can be assumed by measuring the amount of NAIP gene. DNA microarray, oligonucleotide microarray, protein array, northern blotting, RNase protection assay, western blotting, reverse transcription polymerase-chain reaction, etc, can be used for this purpose.

Other terms and concepts used in the present invention are defined in detail in this detailed description of the invention and in the Examples. Based on known literature and such, those skilled in the art could easily and precisely conduct the various techniques performed to carry out the present invention, except for techniques whose sources are specifically indicated. For example, formulations of the pharmaceutical agents in the present invention are described in Remington's Pharmaceutical Sciences, 18^{th} Edition, ed. A. Gennaro, Mack Publishing Co., Easton, Pa., 1990, and such. Genetic engineering and molecular biology techniques are described in Sambrook and Maniatis, in Molecular Cloning-A Laboratory Manual, Cold Spring Harbor Laboratory Press, New York, N.Y., 1989; Ausubel, F. M. et al., Current Protocols in Molecular Biology, John Wiley & Sons, New York, N.Y., 1995, and such.

### BRIEF DESCRIPTION OF DRAWINGS

FIG. 1 is an electrophoretogram indicating changes in NAIP production due to compound 1 (Example 2). Lanes 1 to 5 in the figure show the results of electrophoresis of the samples indicated below.
Lane 1: sample treated with a low concentration of retinoic acid and no candidate compound.
Lane 2: sample treated with a low concentration of retinoic acid followed by addition of a candidate compound.
Lane 3: sample treated with a high concentration of retinoic acid and no candidate compound.
Lane 4: sample treated with a high concentration of retinoic acid followed by addition of a candidate compound.
Lane 5: THP-1 cells

FIG. 2 is a graph showing the cell-death suppressing effect after 1.5 day incubation with a candidate compound when 10 mM solute of compound 1 was used as the candidate compound and menadione was used as a cell-death inducing agent (Example 3).

FIG. 3 is a graph showing the cell suppressing effect after 1.5 day incubation with a candidate compound when 10 mM solution of compound 1 was used as the candidate compound and α-naphthoquinone was used as a cell-death inducing agent (Example 3).

FIG. 4 is a graph showing the cell-death suppressing effect after 1.5 day incubation with a candidate compound when 10 mM solution of compound 1 was used as the candidate compound and 2, 3-dimethoxy-1, 4-naphthoquinone was used as a cell-death inducing agent Example 3).

FIG. 5 is a graph showing the cell-death suppressing effect after 1 day incubation with a candidate compound when 100 μM solution of compound 5 was used as the candidate compound and menadione was used as a cell-death inducing agent (Example 3).

FIG. 6 is a graph showing the cell-death suppressing effect after 2 day incubation with a candidate compound when 100 μM solution of compound 5 was used as the candidate compound and menadione was used as a cell-death inducing agent (Example 3).

FIG. 7 is a graph showing the cell-death suppressing effect after 1 day incubation with a candidate compound when 100 μM solution of compound 6 was used as the candidate compound and menadione was used as a cell-death inducing agent (Example 3).

FIG. 8 is a graph showing the cell-death suppressing effect after 2 day incubation with a candidate compound when 100 μM solution of compound 6 was used as the candidate compound and menadione was used as a cell-death inducing agent (Example 3).

FIG. 9 is a graph showing the cell-death suppressing effect after 1 day incubation with a candidate compound when 100 μM solution of compound 7 was used as the candidate compound and menadione was used as a cell-death inducing agent (Example 3).

FIG. 10 is a graph showing the cell-death suppressing effect after 2 day incubation with a candidate compound when 100 μM solution of compound 7 was used as the candidate compound and menadione was used as a cell-death inducing agent (Example 3).

FIG. 11 is a graph showing the cell-death suppressing effect after 1 day incubation with a candidate compound when 10 μM solution of compound 8 was used as the candidate compound and menadione was used as a cell-death inducing agent (Example 3).

FIG. 12 is a graph showing the cell-death suppressing effect after 2 day incubation with a candidate compound when 10 μM solution of compound 8 was used as the candidate compound and menadione was used as a cell-death inducing agent (Example 3).

FIG. 13 is a graph showing the cell-death suppressing effect after 1.5 day incubation with a candidate compound when 10 mM solution of compound was used as the candidate compound and menadione was used as a cell-death inducing agent (Example 4).

FIG. 14 is a graph showing the cell-death suppressing effect after 1.5 day incubation with a candidate compound when 10 mM solution of compound 1 was used as the candidate compound and menadione was used as a cell-death inducing agent (Example 5)

FIG. 15 is a microscope image of a tissue cross-section of the hippocampus CA1 region of a control Mongolian gerbil, in which compound administration and occlusion were not conducted (Example 6).

FIG. 16 is a microscope image of a tissue cross-section of the hippocampus CA1 region of a control Mongolian gerbil in which the same procedures as Examples 6-1 to 6-3 were conducted, except that the compound was not administered (Example 6).

FIG. 17 is a microscope image of a tissue cross-section of the hippocampus CA1 region of a Mongolian gerbil (Example 6-1).

FIG. 18 is a microscope image of a tissue cross-section of the hippocampus CA1 region of a Mongolian gerbil (Example 6-2).

FIG. 19 is a microscope image of a tissue cross-section of the hippocampus CA1 region of a Mongolian gerbil (Example 6-3).

FIG. 20 is a microscope image of a tissue cross-section of the hippocampus CA1 region of a control Mongolian gerbil, in which the same procedure as in Example 6-4 was conduced except that a compound was not administered (Example 6). The image is of a higher magnification than FIG. 16.

FIG. 21 is a microscope image of a tissue cross-section of the CA1 region of the hippocampus of a Mongolian gerbil (Example 6-4). The image is of the same magnification as FIG. 20.

Table 1 shows the results of using various test compounds to conduct the tests of Example 3.

Table 2 shows the results of testing the therapeutic effect of administrating compound 1 to ALS model mice (Example 7).

### BEST MODE FOR CARRYING OUT THE INVENTION

In the present invention, methods for treating or preventing neurodegenerative diseases comprise administering a compound that upregulates neuronal apoptosis inhibitory protein (NAIP) production. In the present invention, the compounds that upregulate NAIP production comprise one or more compounds selected from a group consisting of: compound 1 or its salts, compound 2 or its salts, compound 3 or its salts, compound 4 or its salts, compound 5 or its salts, compound 6 or its salts, compound 7 or its salts, and compound 8 or its salts as active ingredients.

Moreover, in the present invention, methods of screening for anti-neurodegenerative agents comprise the steps of: (a) contacting a test sample with a cell and measuring NAIP production; and, (b) selecting a compound that increases the NAIP production in comparison with a control test in which the test sample is not contacted with the cell. Furthermore, in the present invention, compounds that can be isolated by the above screening methods may comprise one or more compounds selected from a group consisting of, compound 1 or its salts, compound 2 or its salts, compound 3 or its salts, compound 4 or its salts, compound 5 or its salts, compound 6 or its salts, compound 7 or its salts, and compound 8 or its salts as active ingredients.

Compounds 1 to 8 have the following structural formulas (1) to (8), respectively.

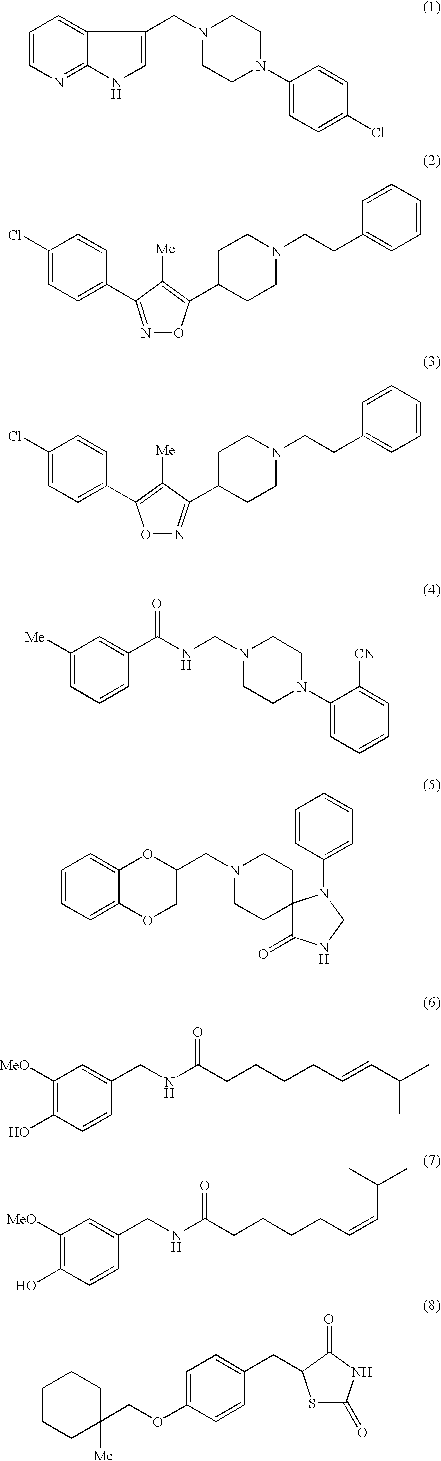

Compounds 1 to 3 are already known as dopamine D4 antagonists; compound 4 is already known as a dopamine D4 agonist; compound 5 is already known as a serotonin 1A receptor antagonist; compounds 6 to 7 are lady known as vanilloid receptor agonists, and compound 8 is already known as a PPAR-γ agonist They are commercially available from, for example, Tocris Cookson Ltd. (England).

Examples of the salts of compounds 1 to 8 include acids (inorganic or organic acids) addition salts, such as hydrochloride, hydrobromide, sulfate, nitrate, acetate, benzoate, maleate, fumarate, succinate, irate, cite, oxalate, methanesulfonate, toluenesulfonate, aspartate, and glutamate. More specifically, for example, salts of compound 1 may be trihydrochloride, salts of compounds 2 and 3 may be monohydrochloride, and salts of compound 4 may be maleate.

Various groups in a compound can be substituted with other groups as long as the biological function of the original compound, such as the dopamine antagonist activity, is retained.

The anti-neurodegenerative agents administered according to the present invention can be the above described active ingredients alone. However, it is preferable to formulate them by mixing with pharmaceutically acceptable carriers, according to the symptoms and administration methods of the pharmaceutical agents. Specifically, the pharmaceutical agents used in the present invention can be mixed with carriers to obtain dosage forms suitable for oral or parenteral administration.

Parenteral administration includes local infusion, intraperitoneal administration, selective intravenous infusion, intravenous injection, subcutaneous injection, organ perfusate infusion, rectal administration, and such. For example, the carriers used for formulation of injectables include sterile water, salt solution, glucose solution, or a mixture of salt solution and glucose, and such. Furthermore, pharmaceutical adjuvants such as buffers, pH controlling agents (disodium hydrogenphosphate, citric acid, and such), isotonizing agents (sodium chloride, glucose, and such), preservatives (methyl paraoxybenzoate, propyl p-hydroxybenzoate, and such), and such can also be comprised. The pharmaceutical agents formulated as above can be sterilized by filtration using sterilizing filters, by mixing the composition with disinfectants, or by irradiating or heating the composition. Alternatively, the agents can be formulated in a powder condition and can be mixed with an above described liquid carrier to prepare an injection solution at the time of use.

The orally administered agents can be formulated into a dosage form suitable for gastrointestinal absorption (for example, tablets, capsules, granules, micro granules, powder, or oral liquid formulations such as suspensions or syrups). Commonly used pharmaceutical adjuvants, for example, binders (syrup, gum arabic, gelatin, sorbit, tragacanth, polyvinylpyrrolidone, hydroxypropylcellulose, and such), excipients (lactose, sugar, corn starch, calcium phosphate, sorbit, glycine, and such), lubricants (magnesia stearate, talc, polyethyleneglycol, silica and such), disintegrants (potato starch, carboxymethylcellulose, and such), moisturizers (sodium laurylsulfate and such) can be used as carriers. Flavors such as strawberry and peppermint can be also added. Moreover, tablets can be coated by common methods. Liquid oral drugs can be solutions or can be used as dried products. Such liquid oral drugs can contain commonly used additives, for example, preservatives (methyl or propyl p-hydroxybenzoate, sorbic acid, and such).

The amount of active component in the pharmaceutical agents can be adjusted according to the extent of the disease and administration method, however, it is usually between 5 and 100% (w/w), and preferably between 10 and 60% (w/w).

The therapeutic methods in the present invention comprise administering a composition, which is an active ingredient of an above described anti-neurodegenerative agent, to a patient who has a neurodegenerative disease. Specifically, the therapeutic methods in the present invention are methods for administering the above described anti-neurodegenerative drugs into patients. Anti-neurodegenerative agents can be administered parenterally (local infusion, intraperitoneal administration, selective intravenous infusion, intravenous injection, subcutaneous injection, organ perfusate infusion, rectal administration, and such) or orally. Moreover, the dose of the pharmaceutical agents varies depending on the age, weight, and symptoms of the patient and the route of administration; however, the amount of the active ingredient can be approximately between 1 and 500 mg/kg.

All publications and patents cited herein are incorporated by reference in their entirety.

### EXAMPLES

Details of the Examples in the present invention are described below. However, the mode for carrying out the present invention is not to be construed as being limited thereto.

### Example 1

(1) 30 μl/ml of solution containing a test compound (10 mM, final concentration of 300 μM was added to RPMI 1640 medium containing THP-1 cells (ATCC TIB202 strain obtained from American Type Culture Collection; 1×10⁶ cells/ml) and 10% fetal bovine serum (FBS). 100 μl/well of the above mixture was aliquoted into each well of a 24 well plate and incubated for tree days at 37° C. The cells were collected, and then lysed in 150 μl of sample lysis solution (composition: 50 mM Tris-HCl (pH 7.5), 150 mM NaCl, and 1% NP-40, into which proteinase inhibitor cocktail (product name: Complete, Roche Diagnostics Corporation) was added according to the attached protocol). The sample solution was recovered by centrifuging the above mentioned cell lysate at 14,000×g, 4° C. for 15 mutes to remove insoluble matter,
(2) Using the sample solution obtained in (1), NAIP concentration in the sample solution was measured using ELISA, according to the method described in JP-A 2000-125861.

The method described in JP-A 2000-125861 is summarized as follows:

### (2-1) Example 1

### Production of the Monoclonal Antibodies

(1) Preparation of the Immunogen

The 1056-2049th region of NAIP cDNA of which nucleotide sequence is shown in SEQ ID NO: 1 was amplified, and the DNA nt (NAIP.256-586) was inserted at the EcoRI site of pGEX-3X (Pharmacia Co.). After confirming the nucleotide sequence, the host Escherichia coli BL21 (DE3) pLysS was transformed by this recombinant vector pGEX-3X(NAIP.256-586) and cultivated in the LB medium for 5 hours at 30° C. Thereafter, IPTG was added to the medium and the cultivation was continued at 20° C. for 3 hours. The bacteria was separated by centrifuging, dissolved into the dissolving solution (PBS, Triton X-100), frozen once at −80° C. and melted, and then subjected to ultrasonic destruction. The product was centrifuged at 1,000×g for 30 minutes, the supernatant was introduced to a glutathione sepharose 4B column so as to pass through it, whereby fusion protein GST-NAIP(256-586) was obtained.

(2) Immunization of the Animal

50 μg/μl of the fusion proteins obtained in the aforementioned (1) was dosed to a Bale/c mouse, intraperitoneally, as the initial immunization. The second immunization was performed 2 weeks after the initial immunization, and immunization was conducted totally six times with one-week interval. At the initial immunization, the fusion protein was dosed in a state in which Freund complete adjuvant of the equal amount was mixed thereto. At the second to fifth immunization, the fusion protein was dosed in a state in which Freund incomplete adjuvant was mixed thereto. At the final immunization, only the fusion protein solution was dosed.

(3) Fusion of Cells

The spleen cells were sterilely isolated three days after the final immunization. The collected spleen cells and the myeloma cell line SP2/0-Ag14 derived from mice were mixed and then subjected to the fusing treatment by using polyethylene glycol #4000. The obtained cells were planted on a 96-well plate, and the fused cells were selected by the HAT culture.

(4) Screening

An ELISA plate on which the NAIP polypeptide used as the immunogen was immobilized and an ELISA plate on which GST was immobilized were prepared. Clones that did not react to the GST plate but reacted only to the NAIP plate were selected and subjected to screening. Next, among the supernatants of the cultures of respective hybridomas, the wells reacted to the NAIP polypeptide were regarded as positive. The cloning of the hybridomas was carried out by using the positive wells in the liming dilution method. The screening process was repeated for the cultures of the hybridomas that were supposed to have only single-type clones, whereby a plurality of hybridomas was obtained.

(5) Production of the Monoclonal Antibodies

Two types of the hybridomas obtund as described above were dosed to a Balb/c mice, intraperitoneally, and the ascites containing the monoclonal antibody was collected after one week. From the collected ascites, the two types of monclonal antibodies hnmc365 and hnmc381 were purified by using an affinity column in which protein G was used.

It was confirmed that the monoclonal antibody hnmc365, produced by hybridoma 656-1 which had been prepared by using fusion protein GST-NAIP(256-586) as the immunogen, belongs to the subclass IgG1 and the epitope thereof is the amino acid sequence of the 354-368th region in SEQ ID NO: 1. It was also confirmed that the monoclonal antibody hnmc381 produced by hybridoma 656-2 belongs to the subclass IgG2b and the epitope thereof is the amino acid sequence of the 373-387th region in SEQ ID NO: 1.

### (2-2) Example 2

### Production of the Polyclonal Antibody

A rabbit (Japanese White Rabbit) was immunized by the standard method, by using as the immunogen the fusion protein GST-NAIP(256-586) prepared in a Or similar to that of example 1(1). The anti-serum was then separated, and the polyclonal antibody was purified by a sepharose 4B column in which the aforementioned fused proteins were bound.

### (2-3) Example 3

### Production of ELISA Kit

(1) Primary Antibody-Immobilized Plate

A solution (20 μg/ml) of the anti-NAIP monoclonal antibody hnmc365 produced in example 1 was dissolved into 10 mmol/l of potassium phosphate buffer (pH 7.5) containing 150 mmol/l of sodium chloride and 1 g/l of sodium azide. 50 μl of this solution was pipetted into each well of a 96-well plate for ELISA. TX plate was stored at 4° C. for 16 hours. Thereafter, the plate was washed with 10 mmol/I potassium phosphate buffer (pH 7.5) contain 150 mmol/l sodium chloride, whereby the plate on which the anti-NAP monoclonal antibody was immobilized was prepared.

(2) Biotinylated Secondary Antibody

0.01 mmol of biotin-amidecaproic acid N-hydroxysuccinic imide ester dissolved into N,N-dimethylformamide was added to 10 mg of the anti-NAIP polyclonal antibody produced in example 2. The mixture was stored at 25° C. for 3 hours and then subjected to dialysis for 16 hours in 50 mmol/l potassium phosphate buffer (pH 7.4), whereby the biotinylated anti-NAIP polyclonal antibody was prepared.

(3) Marker to be Bound to the Secondary Antibody

A solution of horse radish peroxydase-labeled streptoavidin was diluted to the concentration of 0.5 μg/ml with 10 mmol/l potassium phosphate buffer (pH 7.2) containing 150 mmol/l sodium chloride and 1 g/l casein, whereby the marker solution was obtained.

### (2-4) Example 4

### NAIP Assay

(1) Method of Operation

Sample solutions containing the purified NAIP at different concentrations were diluted with 10 mmol/l potassium phosphate buffer (pH 7.2) containing 150 mmol/l sodium chloride. 50 μl of each of the diluted solutions was pipetted into each well of the plate on which the primary antibodies had been immobilized, prepared in example 3(1). The plate was stored at 37° C. for 1 how and then washed off with 10 mmol/l potassium phosphate buffer (pH 7.2) containing 150 mmol/l sodium chloride.

Next, the biotinated anti-NAIP polyclonal antibody prepared in example 3(2) was diluted to the concentration of 0.5 μg/ml with 10 mmol/l potassium phosphate buffer (pH 7.2) containing 150 mmol/l sodium chloride and 1 g/l casein. 100 μl of each of the diluted solutions was pipetted into each well of the aforementioned plate. The plate was stored at 37° C. for 1 hour and then washed off with 10 mmol/l potassium phosphate buffer (H 7.2) containing 150 mmol/l sodium chloride.

As the final step, 100 μl of the solution of horse radish peroxydase-labeled streptoavidin obtained in example 3(3) was pipetted into each well of the aforementioned plate. The plate was stored at 37° C. for 1 hour and then washed off with 10 mmol/l potassium phosphate buffer (pH 72) containing 150 mmol/l sodium chloride.

(2) Color-Developing Reaction and Measurement of Absorbance

3,3′,5,5′-tetramethylbenzidine was dissolved into N,N-dimethylformamide so that the concentration of 3,3′,5,5′-tetramethylbenzidine was 50 mmol/l. The obtained solution was diluted to 1/100 with 100 mmol/l sodium acetate buffer (pH 5.5) and then filed with a filter paper. 0.1 ml of aqueous hydrogen peroxide (10 g/l) was added to 10 ml of the solution, whereby the color developing solution was obtained 50 μl of the color developing solution was pipetted into each well of the aforementioned plate. The plate was stored at 30° C. for 30 minutes. Thereafter, 50 μl of sulfuric acid (2 mol/l) was pipetted into each well of the plate, so that the reaction stopped. Absorbance was then measured at 450 nm.

Specifically, 50 μL of the above described sample solution was added to each well of a plate with immobilized anti-NAIP monoclonal antibody. The immobilized antibody used was anti-NAIP antibody hnmc 841, derived from FERM BP-6921 s (International Patent Organism Depositary accession number, the National Institute of Advanced Industrial Science and Technology (Central 6, 1-1-1 Higashi, Tsukuba, Ibaraki, Japan); deposited on Oct. 9, 1999) which was a hybridoma producing anti-NAIP monoclonal antibody. Measurements were taken three times for each test compound, and average values were calculated. As a control (untreated standard), ELISA was also performed on a sample solution processed as for (1), except that an equal amount of distilled water was added in place of the test compound solution.

(3) As a Result, Over 30 Compounds were Shown to Significantly Increase NAIP Concentration in the Sample Solution and Decided to be Used as a Candidate Compound Hereafter.

### Example 2

(1) 3 ml/well of DMEM-HG medium containing H-SY5Y cells (ATCC CRL 2266 strain obtained from American Type Culture Collection; 1×10⁵ cells/ml), 10% fetal bovine serum (FBS), 100 units/ml penicillin, and 100 μg/ml streptomycin was aliquoted into each well of a 6 well plate, and incubated for two days at 37° C. Either 3 μl/well (lane 2) or 15 μl/well (lane 4) of all-trans retinoic acid (2 mM) was then added to each well. Wells were then incubated for tree days at 37° C.
(2) Next, 30 μl/ml of candidate compound solution (10 mM) was added to each well and incubated for one day at 37° C. After incubation, each well was washed with 0.25 M NaCl, then filled with 10% trichloroacetic acid (TCA) on ice, and kept on ice for ten minutes. Cells were collected with a scrap and centrifuged (14,000 rpm, ten minutes, 4° C.). To prepare the samples for electrophoresis, 100 μl of urea TX solution (9 M urea solution containing 2% Triton X-100 and 5% 2-mercaptoethanol), 25 μl of 10% lithium dodecyl sulfate (LDS) solution, and 2 μl of 2M Tris solution were added to the obtained pellets. The samples were stored at −80° C.
(3) The samples for electrophoresis obtained in (2) were homogenized and then 10 μl of each sample was loaded onto READYGELSJ (Bio Rad Laboratories) and electrophoresed at 120 V for 75 minutes to conduct SDS-PAGE. After electrophoresis, the protein bands were transferred to a Squi-Blot PVDF Membrane (Bio Rad Laboratories) at 100 V for 75 minutes. The membrane was blocked for one day with TBST (Tris buffered saline solution, pH 7.4 containing 0.05% Tween-20 (Sigma)) containing 10% skim mill and then washed with TBST. Primary antibody ME1-3 prepared according to the method described in JP-A Hei 11-116599 and JP-A-2000-125861, identical to biotinylated secondary antibody described in Example 3 (2) in JP-A 2000-125861) was diluted 3000 fold with TBST. The above membrane was immersed in the above primary antibody for two hours at room temperature, washed with TBST, and then immersed for one hour at room temperature in anti-rabbit Ig, horseradish peroxidase (product name; Amersham Pharmacia) that was diluted 3000 fold with TBST. The membrane was developed using a chemiluminescence reagent (ECL-Plus, Amersham Pharmacia) and exposed onto the X-ray film (product name “Bio Max”, Kodak) for a 30 minute exposure time. Furthermore, as a control, analysis was also conducted on samples for electrophoresis prepared in the same way as described above in (1) and (2), except a candidate compound was not added (lanes 1 and 3). Moreover, as a comparison, electrophoresis of THP-1 cell contents was conducted in the same way (lane 5). FIG. 1 shows the results. When compound 1 was used as a candidate compound, samples treated with either low or high concentrations of retinoic acid (lanes 2 and 4) expressed significantly strong 160 kDa bands, presumably derived from NAIP, compared to the controls (lanes 1 and 3).

### Example 3

(1) HeLa cells (ATCC CCL2 strain obtained from American Type Culture Collection; 1×10⁶ cells/ml) were incubated for ten hours at 37° C. under 5% CO₂ in DMEM-HG medium supplemented with 10% FBS, 100 units/ml penicillin, and 100 μg/ml streptomycin in a T75 flask. A 250 μl solution containing a candidate compound (10 μM-10 mM) was added to 25 ml of the above culture and incubated for an additional 1, 1.5, or 2 days at 37° C. under 5% CO₂. Cells were recovered, washed with the above medium, and counted. The cells were resuspended in the above fresh medium so the medium contained 1×10⁵ cells/ml.
(2) 150 μl of the suspension obtained in (1) was added into each well of a 96 well plate and the plate was incubated for four hours at 37° C. A cell-death inducing agent (any one of menadione (sodium bisulfite, 10 mM solution), α-naphthoquinone (10 mM DMSO solution), or 2,3-dimethoxy-1, 4-naphthoquinone (10 mM DMSO solution)) was added to each well and the plate was incubated for four hours at 34° C. Cells were washed with the above medium and 150 μl of the above fresh medium was added to each well and incubated for additional six hours at 37° C. 15 μl of Alamar Blue (product name, BioSource International, Inc. (USA)) was added into each well. After ten hours, luminescence was measured with a luminescence detector to determine the viable cell count at excitation and detection wavelengths of 530 nm and 560 nm, respectively. Moreover, the viable cell count of the control, which was prepared in the same way except that a candidate compound solution was not added “Control”) or DMSO was added instead of a candidate compound “DMSO”), was also determined. An experiment to example how the presence or absence of a candidate compound affected the correlation between the concentration of cell-death inducing agent and viable cell count was conducted by gradually changing the concentration of cell-death inducing agent.

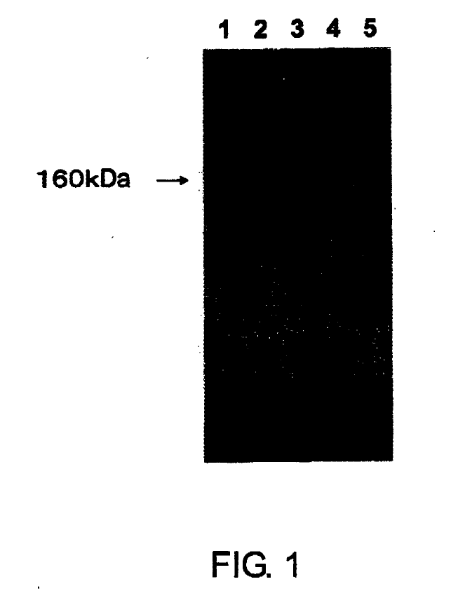

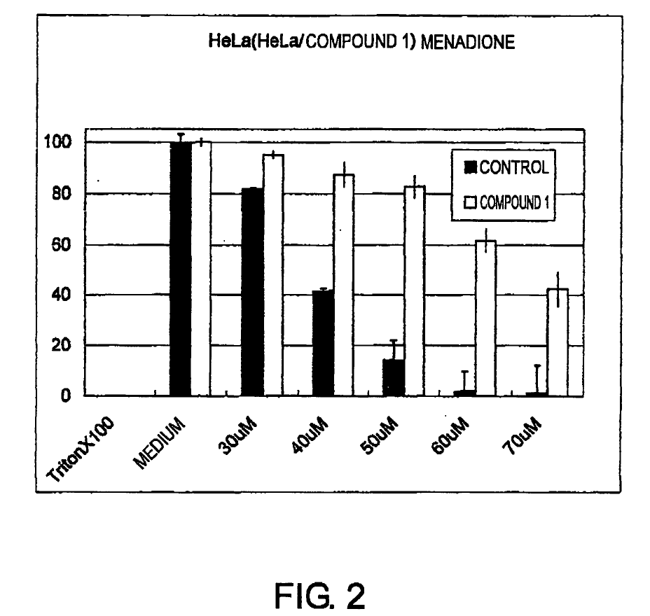

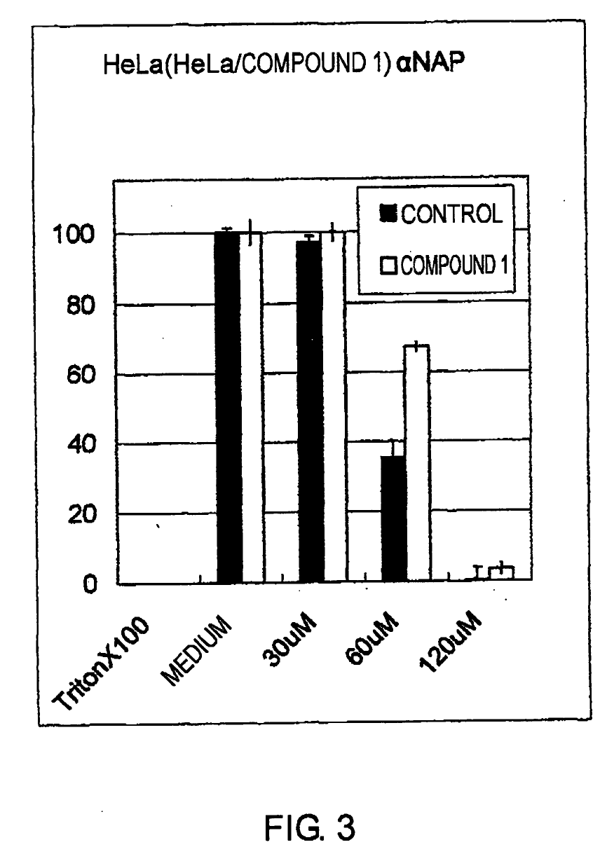

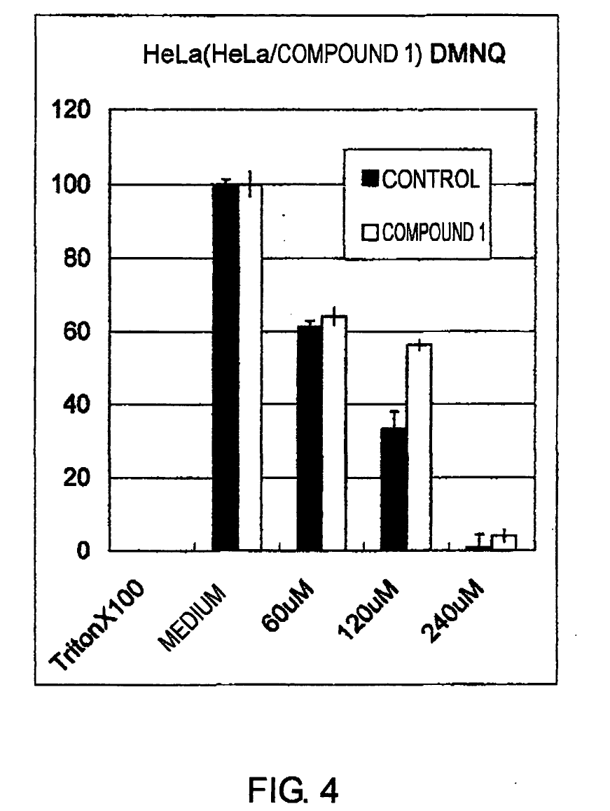

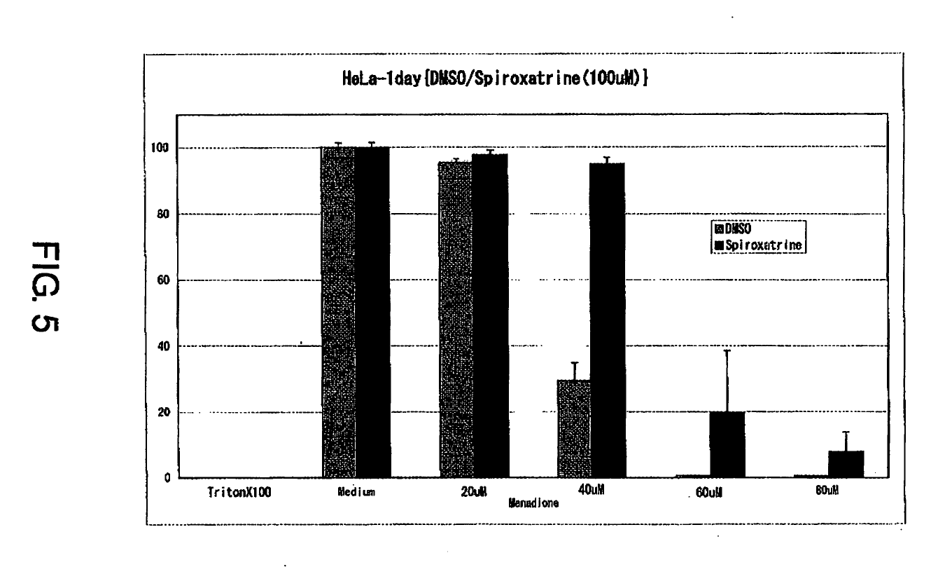

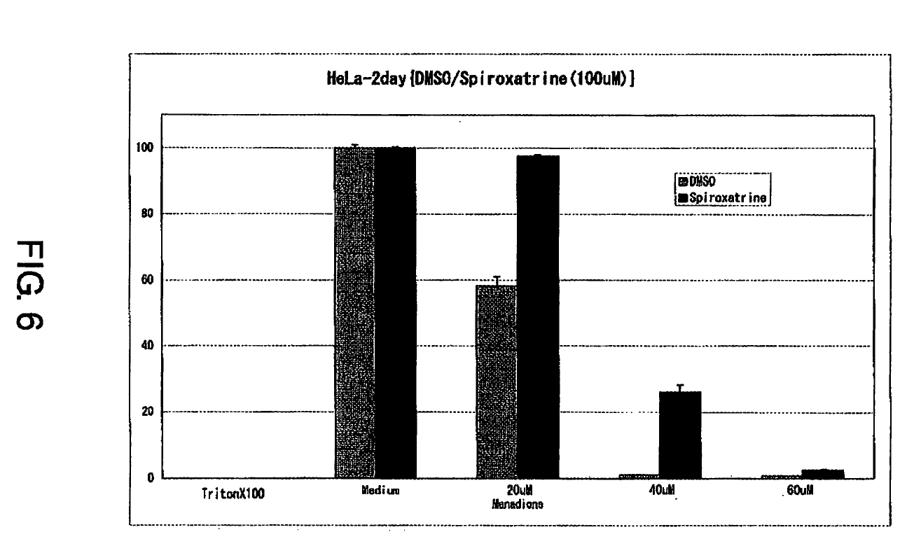

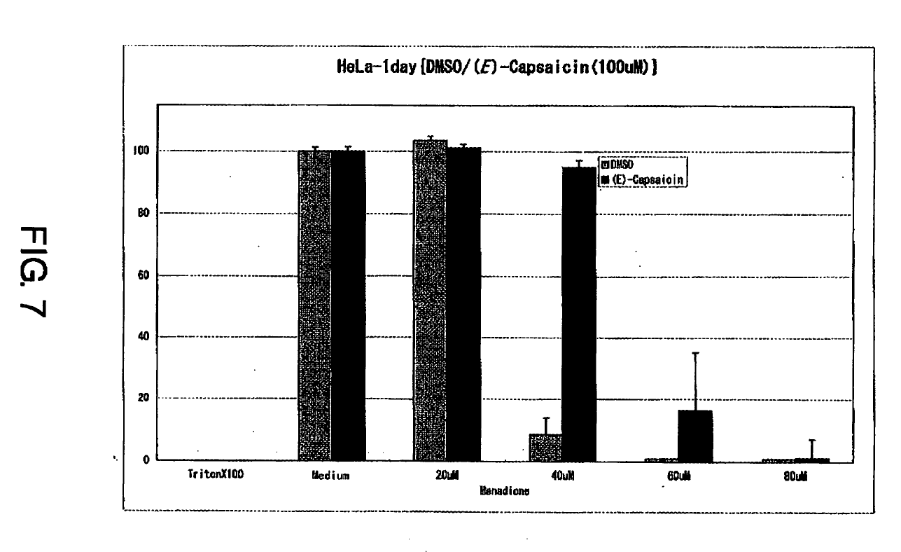

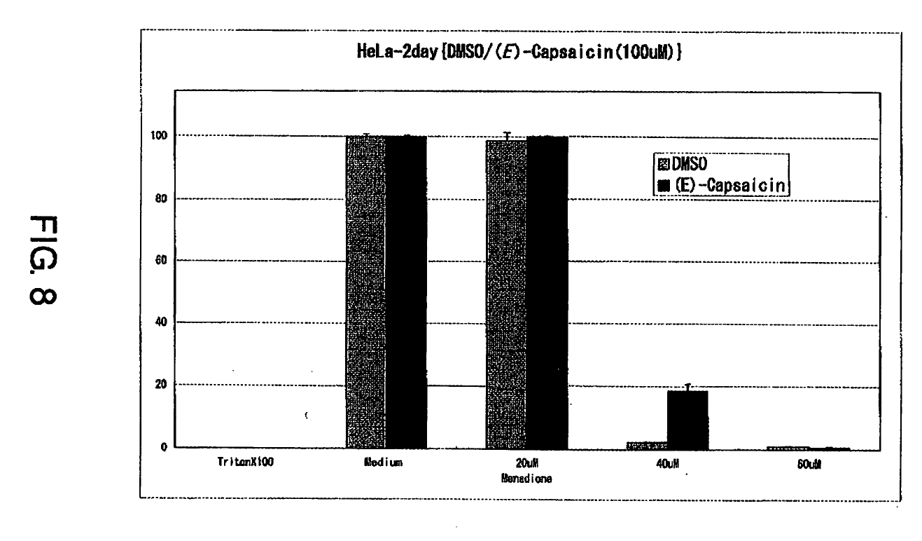

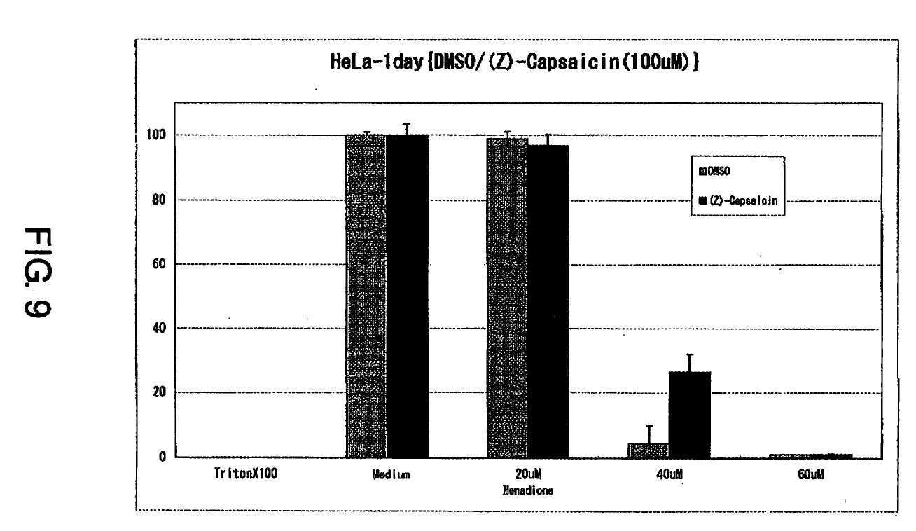

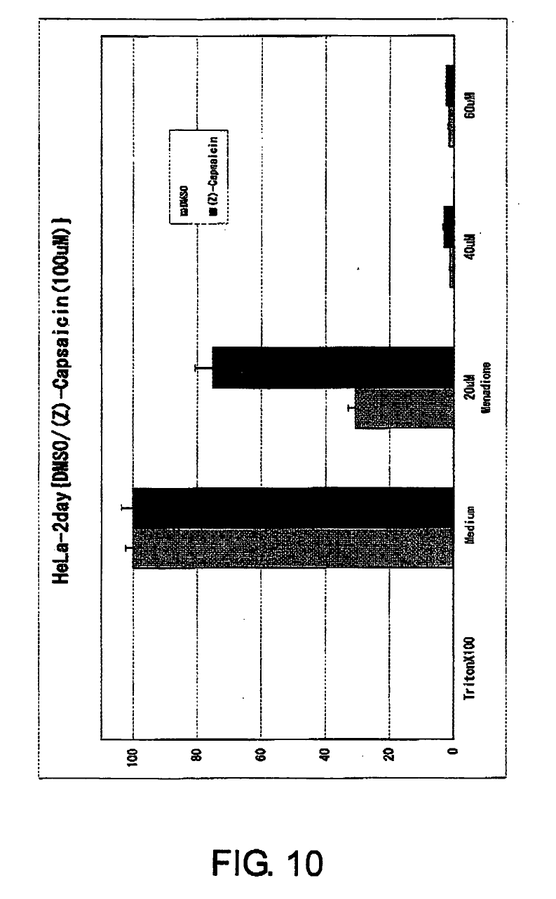

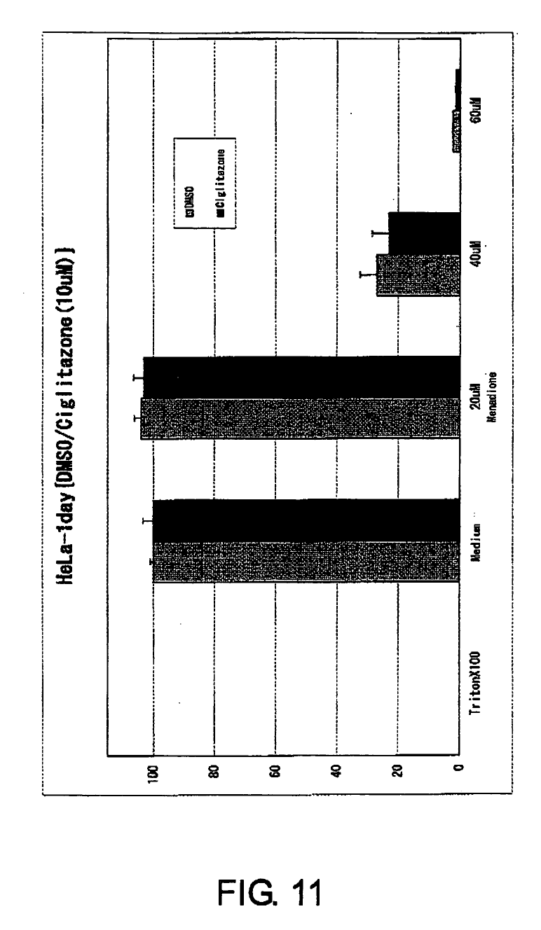

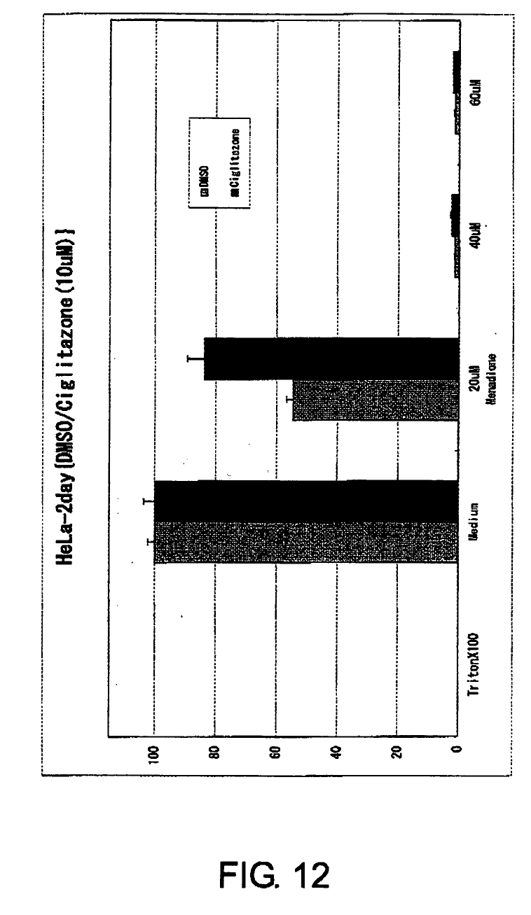

Surprisingly, although the effective concentration varies among the compounds, all the candidate compounds selected in Example 1, the compounds which were shown to significantly increase NAIP concentration in the sample solution, showed cell-death suppressing effect FIGS. 2 to 12 show the results when compounds 1 and 5-8 were used as a candidate compound. The values shown on the horizontal arms indicate the concentration of cell-death inducing agent. The values on the vertical axis indicate relative fluorescence, where zero defines the fluorescence value obtained from a sample prepared in the same way except that 15 μl of 10% Triton X-100 solution was added instead of cell-death inducing agent “Control” and “compound 1” in FIGS. 2 to 4 show results obtained in the absence or presence of compound 1, respectively. “DMSO”, “Spiroxatrine”, “(E)-Capsaicin”, “(Z)-Capsaicin”, and “Ciglitazone” in FIGS. 5 to 12 show results obtained when using DMSO instead of candidate compounds, or using compounds 5, 6, 7, and 8, respectively.

Table 1 shows some of the results obtained by the experiment in the Examples using various test compounds. The experiment shown in Table 1 was conducted using a final concentration of 10 μM of each compound at the time of incubation.

TABLE 1

| Compound | | |
|---|---|---|
| number¹⁾ | Result | Note |
| 1061 | ± | Quinpirole, D2-like agonist |
| 0782 | ± | D4 receptor ligand |
| 1005 | ++ | Compound 3, D4 antagonist |
| 1004 | + or ± | Compound 2, D4 antagonist |
| 1065 | ++ | Compound 4, D4 agonist |
| 1002 | ++++ | Compound 1, D4 antagonist |
| 1003 | = | D2 antagonist |
| 0937 | ± | Pimozide, D2-like antagonist |
| 0524 | ± | D2-like receptor ligand |
| 0701 | ± | 3′-fluorobenzylspiperone, D2-like receptor ligand |

¹⁾Tocris Cooksan Ltd. product number.

### Example 4

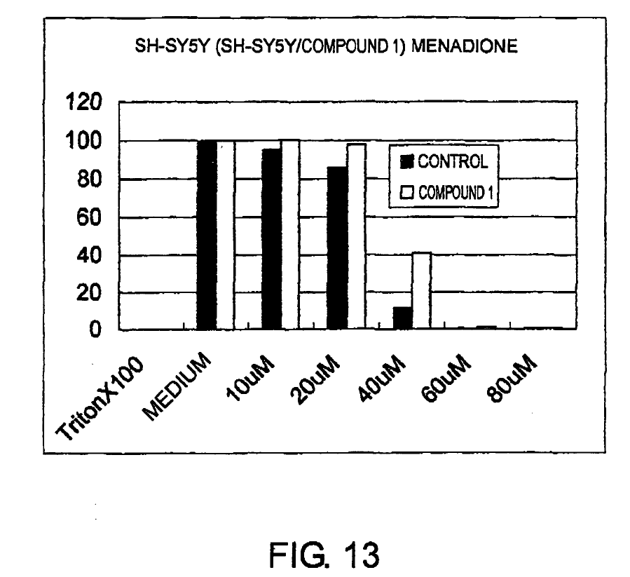

3 ml/well of DMEM-HG medium containing SH-SY5Y cells (1×10⁵ cells/ml), 10% FBS, 100 units/penicillin, and 100 μg/ml streptomycin was aliquoted into each well of a 6 well plate. The plate was incubated for two days at 37° C. and then 15 μl/well of all-trans retinoic acid solution (2 mM) was added into each well. The plate was incubated for three days at 37° C. and hen 30 μl/ml of a candidate compound solution (10 mM) was added to each well. The plate was incubated for one day at 37° C. The cells were recovered and washed with the above medium. Cell number was counted and the cells were rescued in the above fresh medium at 1×10⁵ cells/ml. This suspension was processed as for that in Example 3(2), to examine how the presence or absence of a candidate compound affected the correlation between the concentration of cell-death inducing agent and viable cell count. FIG. 13 shows the results obtained when compound 1 was used as a candidate compound and menadione was used as cell-death inducing agent.

### Example 5

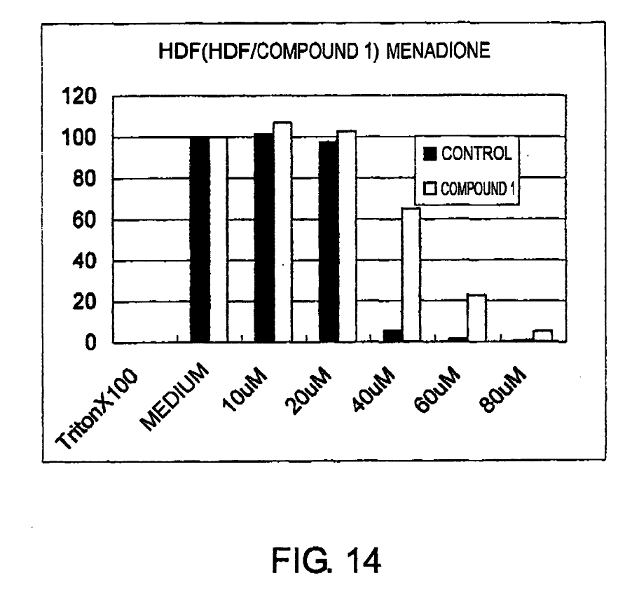

Human fibroblast cells (catalog # 106-05) contained in a normal human fibroblast cell culture kit from Cell Applications, Inc., instead of HeLa cells, were processed as in Example 3 to examine how the presence or absence of a candidate compound affected the correlation between the concentration of cell-death inducing agent and viable cell count. FIG. 14 shows the results obtained when compound 1 was used as a candidate compound and menadione was used as a cell-death inducing agent.

### Example 1-6-4

A candidate compound was dissolved in physiological saline solution to prepare a 100 mM solution with the pH adjusted to 3 to 4 with 1 N NaOH as necessary. This solution was stored. The solution was diluted with physiological saline at time of use to prepare 0.5 ml of a solution containing 8 mg (Example 6-1), 40 mg (Example 6-2), 80 mg (Example 6-3), or 240 mg (Example 6-4) of a candidate compound. The above solution was administered to Mongolian gerbils. In Examples 6-1 to 6-3, 0.5 ml of the above solution was orally administered every 24 hours. In Example 6-4 the solutions was administered once. Furthermore, abnormalities caused by administration were not observed and blood pressure, temperature, and electrocardiograph monitors all showed normal, except for the appearance of catalepsy-like symptoms when the above solution containing 240 mg of a candidate compound was administered twice.

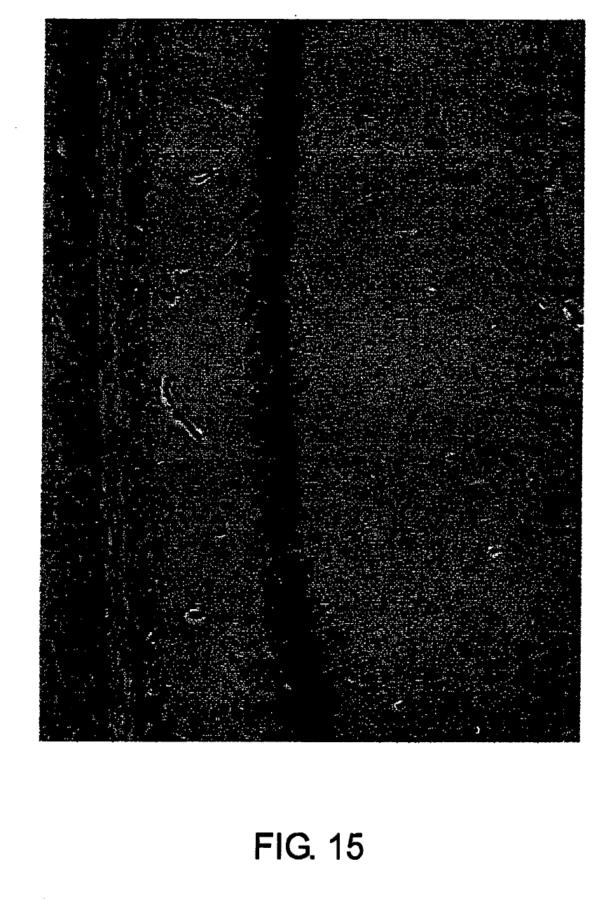

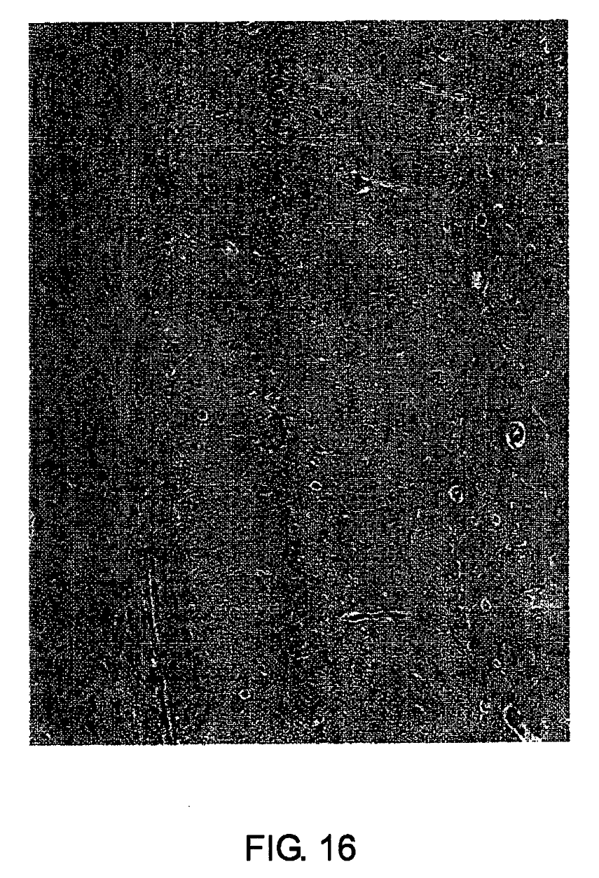

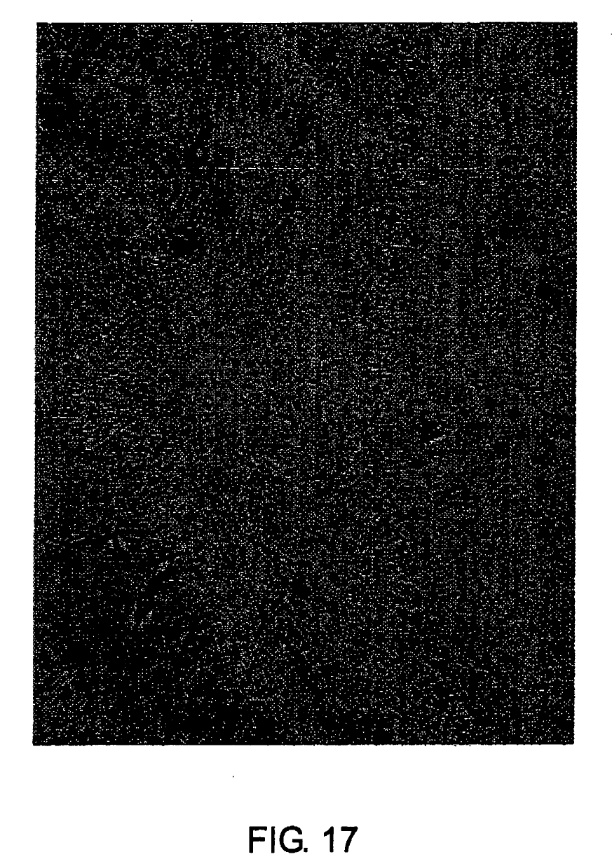

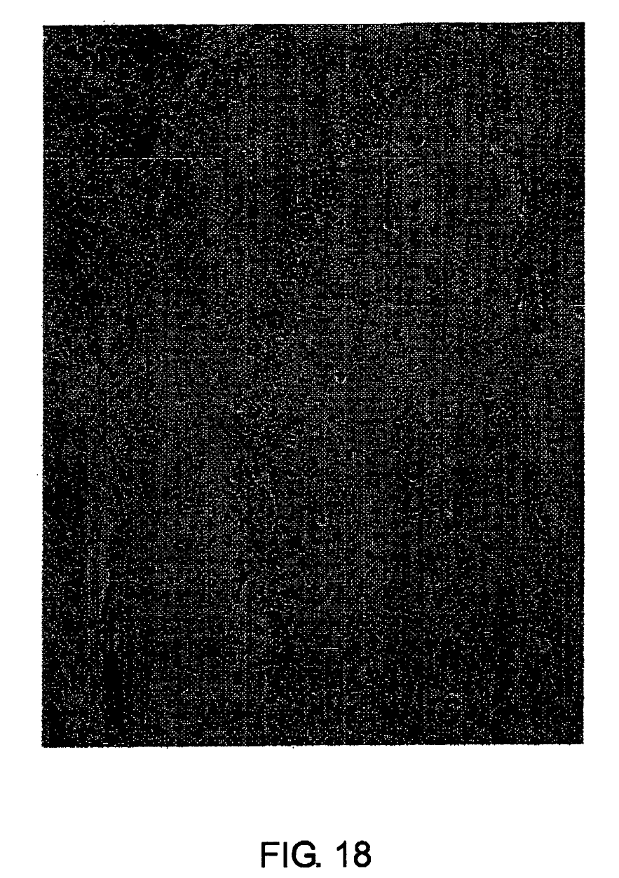

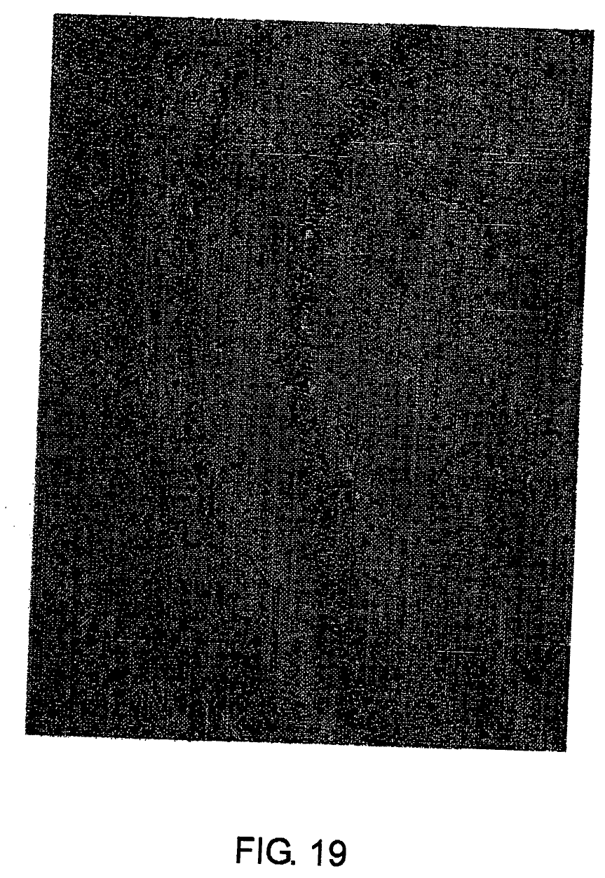

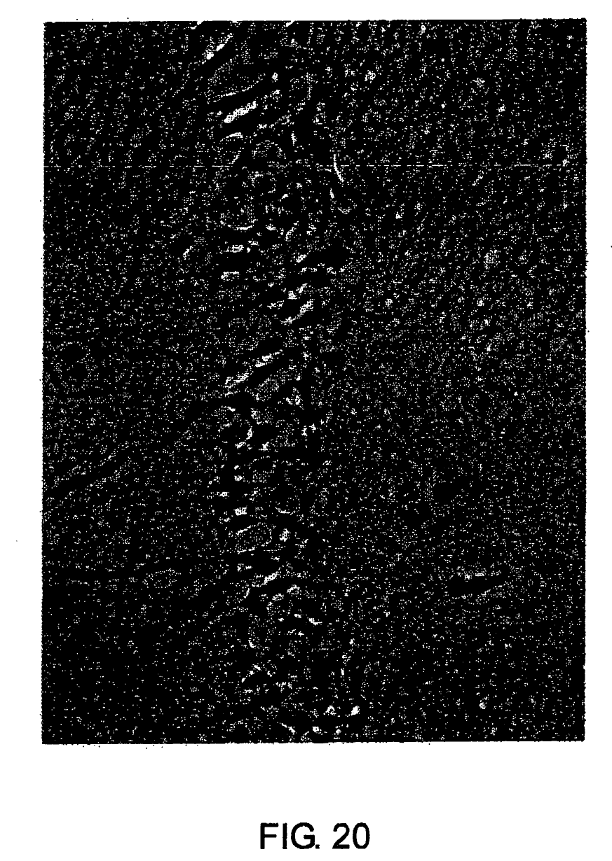

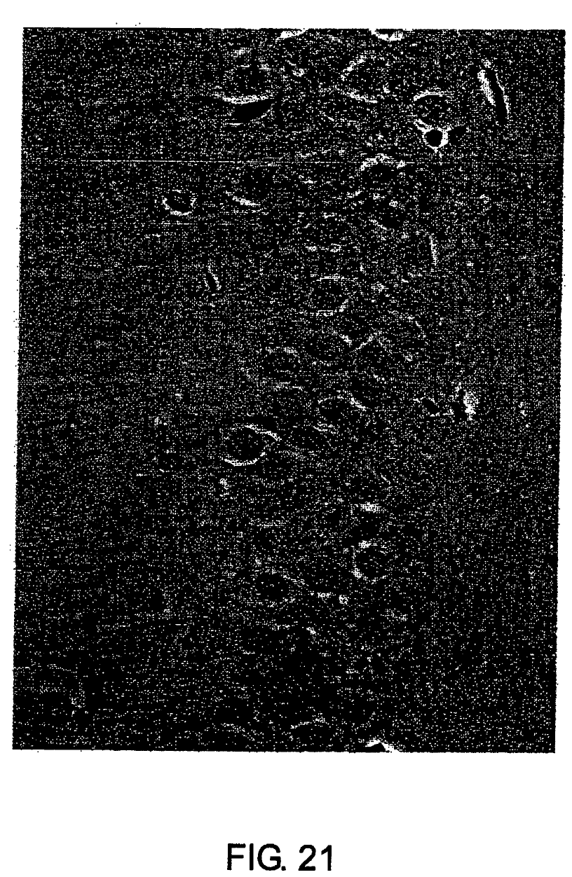

Two hours after the second administration in Examples 6-1 to 6-3, both Mongolian gerbil common carotid arteries were occluded for ten minutes. The gerbils were sacrificed five days later. Two hours after administration in Example 6-4, both Mongolian gerbil common carotid arteries were occluded for ten minutes, and the gerbils were sacrificed three days later. Hematoxylin-eosin stained cross-sections of the brain hippocampus CA1 region tissue were prepared, and the stained sections were expand. Moreover, Mongolian gerbils in which the same process was performed, except that administration of a compound and occlusion were not performed, and other gerbils in which the same process was performed, except that the administration of a compound was not performed, were also sacrificed, and cross-sections were prepared in the same way and used as controls. FIGS. 15 to 21 show the results obtained when compound 1 was used as a candidate compound and the results of the control. As shown in FIGS. 15 to 21, dose-dependent suppression of cell deformation and deciduation in the CA1 region was observed when compound 1 was used as a candidate compound.

### Example 7

The in vivo therapeutic effect of compound 1 was examined using an Amyotrophic Lateral Sclerosis (ALS) model animal.

A Cu/Zn-SOD gene transgenic mouse (Saishin Igaku, Vol. 57 (7), “new Amyotrophic Lateral Sclerosis (ALS) model animals”, July, 2002: p. 1622-1627: obtained from Dr. Masashi Aoki at Tohoku University, School of Medicine, Neurology) was used. The ALS model mice were divided into three groups (physiological saline administered group, compound 1 (8 mg/kg) administered group, compound 1 (40 mg/kg) administered group). Physiological saline, compound 1 (8 mg/kg), and compound 1 (40 mg/kg) were orally administered to each ALS mouse once a day from about 7 days before the predicted day of the symptom development (138-139 days after birth) until the day the mice died.

The results are shown in Table 2, and it was confirmed that administration of compound 1 (40 mg/kg) had an effect in delaying the onset and prolonging the period between the development of symptoms and the death of the ALS model mouse.

TABLE 2

| | | | Days between |
|---|---|---|---|
| | Days before | | symptom |
| | symptom | Days of | development and |
| | development | survival | death |
| Treatment | (P < 0.01) | (P < 0.01) | (P < 0.01) |
| Physiological saline | 139.4 ± 2.4 | 161.6 ± 3.3 | 22.2 ± 1.6 |
| Compound 1 (8 mg/kg) | 138.1 ± 1.9 | 161.7 ± 3.3 | 23.6 ± 2.0 |
| Compound 1 (40 mg/kg) | 145.2 ± 1.8 | 174.6 ± 2.4 | 28.9 ± 1.6 |

### INDUSTRIAL APPLICABILITY

As described above in detail, when administered to subjects, the anti-neurodegenerative agents administered according to the present invention comprise the effect of increasing NAIP production, and further of suppressing neurodegeneration. Therefore, the anti-generative agents used in the present invention are useful for eating and preventing neurodegenerative diseases such as amyotrophic lateral sclerosis (ALS), spinal muscular atrophy (SMA), Huntington's disease, Parkinson's disease, Alzheimer's disease, cerebrospinal paralysis accompanied by injury and cerebral vascular disorder, dementia after cerebral vascular disorder, and dementia accompanied by other neuronal degeneration.

1. A method for treating or preventing a neurodegenerative disease comprising administering a compound that upregulates neuronal apoptosis inhibitory protein (NAIP) production.

2. The method of claim 1, wherein the compound that upregulates neuronal apoptosis inhibitory protein (NAIP) production is selected from the group consisting of: a dopamine receptor antagonist, a serotonin receptor antagonist, a vanilloid receptor agonist, a peroxisome proliferators-activated receptor (PPAR)-γ agonist, and a combination thereof.

3. The method of claim 2, wherein the dopamine receptor antagonist is a dopamine D4 antagonist.

4. The method of claim 1, wherein the compound that upregulates neuronal apoptosis inhibitory protein (NAIP) production is selected from the group consisting of: a dopamine D4 antagonist, a dopamine D4 agonist, a serotonin 1A antagonist, and a combination thereof.

5. The method of claim 3, wherein the dopamine D4 antagonist is selected from the group consisting of: 3-[4-(4-chlorophenyl) piperazin-1-yl]methyl]-1H-pyrrolo[2,3-b]pyridine or salts thereof, 5-(4-chlorophenyl)-4-methyl-3-(1-(2-phenylethyl)piperidin-4-yl) isoxazole or salts thereof, 3-(4-chlorophenyl)-4-methyl-5-(1-(2-phenylethyl)piperidin-4-yl) isoxazole or salts thereof, and a combination thereof.

6. The method of claim 4, wherein the dopamine D4 agonist is N-methyl-4-(2-cyanophenyl) piperazinyl-3-methylbenzamine or salts thereof.

7. The method of claim 4, wherein the serotonin 1A antagonist is 8-[(2,3-Dihydro-1,4-benzodioxin-2-yl)methyl]-1-phenyl-1,3,8-triazaspiro[4,5]decan-4-one (spiroxatrine) or salts thereof.

8. The method of claim 2, wherein the vanilloid receptor agonist is selected from the group consisting of: (EN-[(4-Hydroxy-3-methoxyphenyl)methyl]-8-methyl-6-nonenamide (E-capsaicin) or salts thereof, (Z)-N-[((4Hydroxy-3-methoxyphenyl)methyl]-8-methyl-6-nonenamide (Z-capsaicin) or salts thereof and a combination thereof.

9. The method of claim 2, wherein the peroxisome proliferators-activated receptor (PPAR)-γ agonist is 5-[[4-[(1-Methylcyclohexyl)methoxy]phenyl]methyl]-2,4-thiazolidinedione (ciglitazone) or salts thereof.

10. A method for treating or preventing a neurodegenerative disease comprising administering one or more compounds selected from the group consisting of 3-[4-(4-chlorophenyl) piperazin-1-yl]methyl]-1H-pyrrolo[2,3-b]pyridine or salts thereof, 5-(4-chlorophenyl)-4-methyl-3-(1-(2-phenylethyl)piperidin-4-yl) isoxazole or salts thereon 3-(4-chlorophenyl)-4-methyl-5-(1-(2-phenylethyl) piperidin-4-yl) isoxazole or salts thereof, N-methyl-4-(2-cyanophenyl) piperazinyl-3-methylbenzamine or salts thereof, 8-[(2,3-Dihydro-1,4-benzodioxin-2-yl)methyl]-1-phenyl-1,3,8-triazaspiro[4,5]decan-4-one or salts thereof, (E)-N-[(4-Hydroxy-3-methoxyphenyl)methyl]-8-methyl-6-nonenamide or salts thereof, (Z)-N-[(4-Hydroxy-3-methoxyphenyl)methyl]-8-methyl-6-nonenamide or salts thereof, and 5-[[4-[(1-Methylcyclohexyl)methoxy]phenyl]methyl]-2,4-thiazolidinedione or salts thereof.

11. A method of screening for an anti-neurodegenerative agent, comprising the steps of:
(a) contacting a test sample with the cell and measuring neuronal apoptosis inhibitory protein (NAIP) production; and,
(b) selecting a compound that increases the NAIP production in comparison with a control test in which the test sample is not contacted with the cell.

12. The method of claim 11, wherein the neuronal apoptosis inhibitory protein (NAIP) production is measured by DNA microarray, oligonucleotide microarray, protein array, northern blotting, RNase protection assay, western blotting, or reverse transcription polymerase-chain reaction.

13. A compound that upregulates neuronal apoptosis inhibitory pin (NAIP) production, wherein the compound can be isolated by the method of claim 11 or 12.
